(19) United States
(12) Patent Application Publication
(10) Pub. No.: US 2021/0169871 A1
(43) Pub. Date: Jun. 10, 2021

(54) PHARMACEUTICAL COMPOSITIONS WITH REDUCED TERT-BUTANOL LEVELS

(71) Applicant: TARVEDA THERAPEUTICS, INC., Watertown, MA (US)

(72) Inventors: Mark T. BILODEAU, Waltham, MA (US); Eugene ZHOROV, Marblehead, MA (US); Rajesh R. SHINDE, Lexington, MA (US); Christopher SEARS, Belmont, MA (US); Samantha BRADY, Watertown, MA (US); Beata SWERYDA-KRAWIEC, Marlborough, MA (US)

(73) Assignee: Tarveda Therapeutics, Inc., Watertown, MA (US)

(21) Appl. No.: 17/045,325

(22) PCT Filed: Apr. 3, 2019
(86) PCT No.: PCT/US2019/025526

§ 371 Date: Oct. 5, 2020

Related U.S. Application Data
(60) Provisional application No. 62/653,118, filed on Apr. 5, 2018.

(51) Int. Cl. A61K 31/4745; A61K 9/19; A61K 47/10

(52) U.S. Cl. CPC A61K 31/4745; A61K 47/10; A61K 9/19

(57) ABSTRACT

The present invention provides improved formulation with reduced tert-butanol levels and/or manufacturing process for drug compounds that form a solvate with tert-butanol. For example, the drug compound may be SDC-TRAP-0063.

### REFERENCE TO RELATED APPLICATIONS

The present application claims priority to U.S. Provisional Patent Application No. 62/653,118, filed Apr. 5, 2018, entitled PHARMACEUTICAL COMPOSITIONS WITH REDUCED TERT-BUTANOL LEVELS, the contents of which are herein incorporated by reference in their entirety.

### FIELD OF THE DISCLOSURE

The present invention generally relates to formulation and production of pharmaceutical compositions with reduced tert-butanol (TBA) levels.

### BACKGROUND

tert-Butyl Alcohol (tert-butanol or TBA) is a common solvent used in manufacturing and formulation of pharmaceutical compositions. It is used to aid active pharmaceutical ingredient (API) dissolution, shorten lyophilization cycles, or improve lyophilization cake quality. TBA levels in pharmaceutical compositions have be limited to the extent possible to meet product specifications, good manufacturing practices, or other quality-based requirements. There remains a need for a composition and/or a method suitable of reducing TBA levels.

### SUMMARY OF THE DISCLOSURE

The present application provides a method for TBA removal for a drug compound which entraps TBA and/or forms a solvate with TBA, comprising adding at least one polyol prior to the lyophilization process. The drug compound may have high pH and/or limited solubility in water. The present application also provides a pharmaceutical composition comprising a drug compound and a reduced amount of TBA achieved via adding polyol(s) and/or polyether(s) and not exclusively by lyophilization cycle modifications. Methods of making and using the pharmaceutical composition are also provided.

The present invention is described in further detail by the figures and examples below, which are used only for illustration purposes and are not limiting.

Other features and advantages of the instant invention will be apparent from the following detailed description and claims.

### DETAILED DESCRIPTION

For drug compounds with limited solubility in water including drugs that require high pH for solubility, TBA may be selected as a solvent during the manufacturing process. In a few instances, TBA can be reduced to acceptable levels during lyophilization process via annealing, modifications of the freeze-drying cycles, or secondary drying, but there is no reliable solution for TBA removal for drug compounds which entrap TBA and/or form solvates with TBA.

The present application provides a method for TBA removal during the lyophilization process that can be used for a drug compound that entraps TBA and/or forms a solvate with TBA, wherein TBA is used in the manufacturing process of the drug compound. The drug compound may have solubility only at high pH and/or limited solubility in water at any pH. The method comprises adding at least a polyol, such as diols or triols including propylene glycol, glycerol and various molecular weight polyethers such as polyethylene glycols, as excipients to the formulation, wherein the polyol or polyether forms a solvate with the compound and replaces TBA in the lyophilized drug product.

Solvate, as used herein, refers to a non-covalent association between a solvent and a dissolved drug compound.

Polyol or polyether, as used herein, refers to an organic molecule that has at least two hydroxyl (—OH) groups or at least two ether groups. Diol, as used herein, refers to an organic molecule that has two hydroxyl groups. Non-limited examples of polyols and polyethers include propylene glycols (PG) and polyethylene glycols ((PEG) such as PEG-400 and PEG-1000, PEG-2000 and PEG-4000). Triol, as used herein, refers to an organic molecule that has three hydroxyl groups. Non-limited examples of triols include glycerol.

At least one polyol or polyether, such as a diol, triol or PEG may be added to a bulk drug solution containing an alkaline co-solvent system comprising TBA and at least one other solvent, before the lyophilization process. The co-solvent system comprises TBA and at least one other solvent such as but not limited to water, ethanol, n-propanol, n-butanol, isopropanol, methanol, acetone, ethyl acetate, dimethyl carbonate, acetonitrile, dichloromethane, methyl ethyl ketone, methyl isobutyl ketone, 1-pentanol, methyl acetate, carbon tetrachloride, dimethyl sulfoxide, hexafluoroacetone, chlorobutanol, dimethyl sulfone, acetic acid, and cyclohexane.

In some embodiments, the process of reducing TBA in a drug compound comprises the steps of:

1). dissolving a drug compound in an alkaline co-solvent system comprising tert-butanol (TBA) and at least one other solvent to obtain a drug solution;

2). adding at least one polyol or polyether, such as a diol, triol or PEG, to the drug solution to obtain a pre-lyophilization mixture; and

3). conducting lyophilization to obtain dry powders of the drug compound (drug product).

The co-solvent system in step 1) may comprise a target concentration of about 5% to about 50% (e.g., about 10%, 20%, 25%, 30%, or 40%) by weight of TBA. The other solvent in the co-solvent system in step 1) may be any suitable solvent, such as but not limited to, water, ethanol, n-propanol, n-butanol, isopropanol, methanol, acetone, ethyl acetate, dimethyl carbonate, acetonitrile, dichloromethane, methyl ethyl ketone, methyl isobutyl ketone, 1-pentanol, methyl acetate, carbon tetrachloride, dimethyl sulfoxide, hexafluoroacetone, chlorobutanol, dimethyl sulfone, acetic acid, and cyclohexane.

Optionally, the mixture from step 2) may be filtered before lyophilization. The mixture in step 2) may be contained in vials. Optional aseptic vial filling may also be conducted before lyophilization. The vials can be sealed after lyophilization in step 3).

The drug solution obtained in step 1) may comprise about 0.05 mol/L, 0.10 mol/L, 0.15 mol/L, 0.20 mol/L, 0.30 mol/L, 0.40 mol/L, 0.50 mol/L of the drug compound. The solution may have a pH of at least about 4, 5, 6, 7, 8, 9, 10, 11, or 12.

The dry powders obtained in step 3) may comprise at least 20%, 25%, 50%, 75%, 90%, or 95% by weight of the drug compound. The dry powders obtained in step 3) may comprise less than about 5.0%, 4.5%, 4.0%, 3.5%, 3.0%, 2.5%, 2.0%, 1.0%, 0.5%, or 0.1% by weight of TBA. In some embodiments, the dry powders obtained in step 3) may comprise between about 5% and about 0.1%, between about 4.0% and about 0.1%, between about 3.0% and about 0.1%, between about 2.0% and about 0.1%, between about 1.0% and about 0.1%, or between 0.5% and about 0.1% by weight of TBA.

The dry powders can be resolved in a solvent to obtain a pharmaceutical composition to be administered to a patient. Such a pharmaceutical composition may have less than about 5.0%, 4.5%, 4.0%, 3.5%, 3.0%, 2.5%, 2.0%, 1.0%, 0.5% or 0.1% of TBA by weight and at least about 25 mg/mL, 50 mg/mL, 75 mg/mL, 100 mg/mL, 125 mg/mL, 150 mg/mL, 200 mg/mL, 225 mg/mL, or 250 mg/mL of the drug compound, its tautomer, or its pharmaceutically acceptable salt. In some embodiments, the pharmaceutical composition may comprise between about 5% and about 0.1%, between about 4.0% and about 0.1%, between about 3.0% and about 0.1%, between about 2.0% and about 0.1%, between about 1.0% and about 0.1%, or between 0.5% and about 0.1% by weight of TBA.

In some embodiments, in step 2), the polyol or polyether is selected from the group consisting of PG, glycerol, and PEG. The target concentration of the polyol in the pre-lyophilization mixture may be between about 1% to about 10% by weight, e.g., about 1% to about 5%; about 3%; about 4%; about 5%, or about 6%. The resulting lyophilized powder of SDC-TRAP-0063 will contain from 10 to 100 w/w % of PEG or another polyol.

In some embodiments, in step 2), PEG is added to the drug solution. In one embodiment, 3-4% of PEG is added. PEG may be PEG400 (i.e., PEG-400), PEG-1000 (i.e., PEG-1000), PEG2000 (i.e., PEG-2000), or PEG4000 (i.e., PEG-4000). The number in a PEG molecule name, as used herein, refers the approximate average molecular weight of the PEG molecule. Molecular weight, as used herein, generally refers to the mass or average mass of a material. In practice, the molecular weight of polymers and oligomers can be estimated or characterized in various ways including gel permeation chromatography (GPC) or capillary viscometry. GPC molecular weights are reported as the weight-average molecular weight (Mw) as opposed to the number-average molecular weight (Mn). Capillary viscometry provides estimates of molecular weight as the inherent viscosity determined from a dilute polymer solution using a particular set of concentration, temperature, and solvent conditions.

In some embodiments, the lyophilization process in step 3) may be a standard lyophilization process. The lyophilization may comprise steps of freezing, annealing, primary drying, and secondary drying.

In some embodiments, the lyophilization process in step 3) may comprise very slow drying cycles. Slow and/or gentle drying cycles may be used to get lyophilized drug product without step 2), i.e., without adding any polyol to the drug solution obtained in step 1). In some embodiments, the primary drying may be at least 5 hours, 8 hours, 10 hours, or 20 hours. The secondary drying may be at least 5 hours, 8 hours, 10 hours, or 20 hours. Any lyophilization process in WO2006076620, the contents of which are incorporated herein by reference in their entirety, may be used. For example, the lyophilization may comprise steps of: freezing the pre-lyophilization mixture to a temperature below about −40° C. to form a frozen solution; holding the frozen solution at or below −40° C. for at least 2 hours; ramping the frozen solution to a primary drying temperature between about −40° C. and about 0° C. to form a dried solution; holding for about 10 to about 70 hours; ramping the dried solution to a secondary drying temperature between about 25° C. and about 40° C.; and holding for about 5 to about 40 hours. In another example, the lyophilization may comprise steps of: freezing the pre-lyophilization mixture to about −50° C. to form a frozen solution; holding the frozen solution at about −50° C. for at least 2 hours to about 4 hours; ramping to a primary drying temperature between about −20° C. and about −12° C. to form a dried solution; holding at a primary drying temperature for about 10 to about 48 hours; ramping the dried solution to a secondary drying temperature between about 25° C. and about 40° C.; and holding at a secondary drying temperature for at least 5 hours up to about 20 hours. In some embodiments, the pressure may be about 150 microns throughout primary drying and about 50 microns throughout secondary drying. In yet another example, the lyophilization may comprise steps of: freezing from +25° C. to −50° C. in about 8 hours; holding at −50° C. for about 5 hours; warming and drying from −50° C. to −25° C. in about 7 hours; holding for about 20 hours at −25° C.; warming and drying from −25° C. to −15° C. in about 2 hours and holding for about 20 hours at −15° C.; warming and drying from −15° C. to 40° C. in about 6 hours and holding for about 20 hours at 40° C. A chamber pressure of 150 microns is maintained throughout drying.

### Drug Compounds

In some embodiments, the drug compound of the present disclosure has a high pH, such as pH >about 7, 8, 9, 10, 11, or 12. A pH value is a measure of ions within a solution. It is the concentration of hydrogen ions in a solution. A solution with a high concentration of hydrogen ions is acidic. A solution with a low amount of hydrogen ions is basic, or also known as alkaline. The drug compound in the present disclosure is soluble at basic pH. The pH of a solution containing this compound can be measured by a pH meter, various types of chemical pH indicators, or by acid-base titration.

In some embodiments, the drug compound of the present disclosure has a low solubility in water. Water solubility is measured in terms of the maximum amount of a compound in water at equilibrium, i.e., the amount of a compound in a saturated solution in water. According to the present disclosure, there is less than about 0.3 moles, 0.2 moles, 0.1 mole, or 0.05 mole of drug compound in 1 liter of water.

In some embodiments, the drug compound in solution has a high pH and low solubility in water.

In some embodiments, the drug compound drug compound entraps TBA and/or forms a solvate with TBA.

In some embodiments, the drug compound is SDC-TRAP-0063, its tautomer, or its pharmaceutically acceptable salt. It is described in PCT Application No. PCT/US2013/036783 and has the following structure:

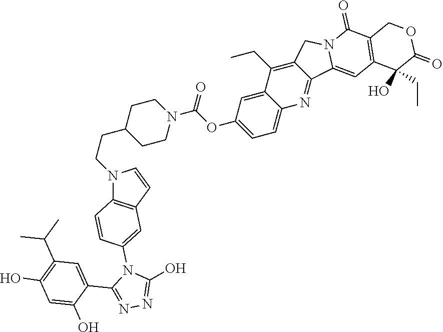 [SMILES: CCc1c2c(nc3ccc(OC(=O)N4CCC(CCn5ccc6cc(-n7c(O)nnc7-c7cc(C(C)C)c(O)cc7O)ccc65)CC4)cc13)-c1cc3c(c(=O)n1C2)COC(=O)[C@]3(O)CC]

((S)-4,11-diethyl-4-hydroxy-3,14-dioxo-3,4,12,14-tetrahydro-1H-pyrano[3′,4′:6,7]indolizino[1,2-b]quinolin-9-yl 4-(2-(5-(3-(2,4-dihydroxy-5-isopropylphenyl)-5-hydroxy-4H-1,2,4-triazol-4-yl)-1H-indol-1-yl)ethyl)piperidine-1-carboxylate); Formula C₄₉H₄₉N₇O₉; molecular weight 880.

In solution, SDC-TRAP-0063 contains a lactone ring at pH-dependent equilibrium with the corresponding open chain carboxylic acid form. At high pH (above pH of around 9) the equilibrium shifts toward an open ring carboxylic acid form and at low pH it shifts toward the closed ring lactone form.

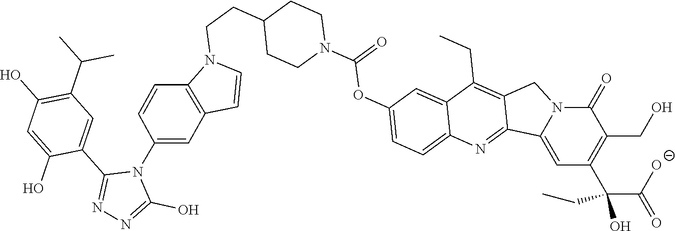 [SMILES: CCc1c2c(nc3ccc(OC(=O)N4CCC(CCn5ccc6cc(-n7c(O)nnc7-c7cc(C(C)C)c(O)cc7O)ccc65)CC4)cc13)-c1cc([C@@](O)(CC)C(=O)[O-])c(CO)c(=O)n1C2]

The open ring carboxylic acid form may form a salt with cationic ions include, but not limited to, lithium, aluminum, calcium, magnesium, potassium, sodium, zinc, barium, bismuth, benethamine, diethylamine, tromethamine, benzathid, chloroprocaine, choline, diethanolamine, ethylenediamine, meglumine, or procaine.

The sodium salt (SDC-TRAP-0063 Sodium or SDC-TRAP-0063 Na) of the carboxylic acid derivative has a structure of

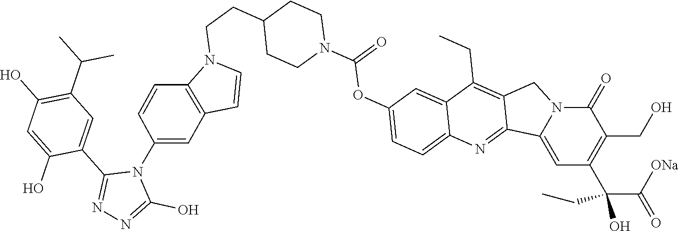

(Sodium (S)-2-(2-((4-(2-(5-(3-(2,4-dihydroxy-5-isopropylphenyl)-5-hydroxy-4H-1,2,4-triazol-4-yl)-1H-indol-1-yl)ethyl)piperidine-1-carbonyl)oxy)-12-ethyl-8-(hydroxymethyl)-9-oxo-9,11-dihydroindolizino[1,2-b]quinolin-7-yl)-2-hydroxybutanoate) or its tautomer:

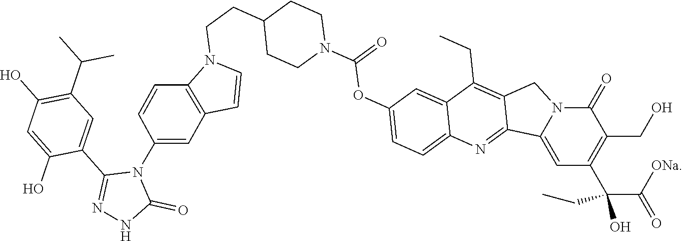

Structures of SDC-TRAP-0063 in both lactone and sodium salt form:

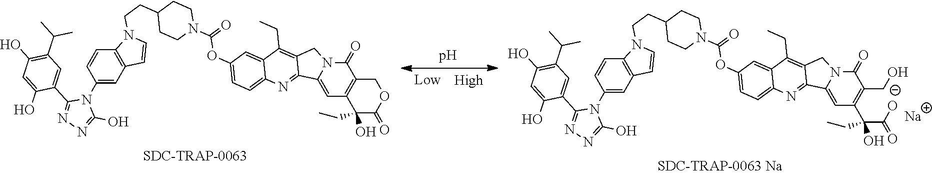 [SMILES: CCc1c2c(nc3ccc(OC(=O)N4CCC(CCn5ccc6cc(-n7c(O)nnc7-c7cc(C(C)C)c(O)cc7O)ccc65)CC4)cc13)-c1cc3c(c(=O)n1C2)COC(=O)[C@]3(O)CC ; CCc1c2c(nc3ccc(OC(=O)N4CCC(CCn5ccc6cc(-n7c(O)nnc7-c7cc(C(C)C)c(O)cc7O)ccc65)CC4)cc13)-c1cc([C@@](O)(CC)C(=O)[O-])c(CO)c(=O)n1C2 ; [Na+]]

During the manufacturing process, SDC-TRAP-0063 is converted to SDC-TRAP-0063 Sodium, which is the dominant form at pH above approximately 9.3. SDC-TRAP-0063 Sodium drug product is aseptically manufactured as a sterile-filtered solution that is lyophilized. TBA is used as a solvent during the manufacturing process, because SDC-TRAP-0063 does not have good solubility in water. There is minimal impact from annealing or secondary drying modifications on TBA level in SDC-TRAP-0063 Sodium drug product.

Not willing to be bound to any theory, SDC-TRAP-0063 Sodium forms solvates with TBA and it requires a stronger solvating agent to break TBA solvates. Common diols and polyethers, such as propylene glycols (PG), Polyethylene glycols ((PEG) such as PEG-400, PEG-1000, PEG-2000, or PEG-4000) and triols (Glycerol) added as excipients to the formulation can form a solvate and replace TBA in the lyophilized product.

A process of producing dry powders comprising SDC-TRAP-0063 or a tautomer or a pharmaceutically acceptable salt thereof, comprising the steps of:

1). dissolving SDC-TRAP-0063 in an alkaline co-solvent system comprising tert-butanol (TBA) and at least one other solvent to obtain a drug solution;

2). adding at least one polyol or polyether, such as a diol, triol or PEG, to the drug solution to obtain a mixture; and

3). conducting lyophilization to obtain the dry powders.

The other solvent in step 1) may be any suitable solvent, such as but not limited to, water, ethanol, n-propanol, n-butanol, isopropanol, methanol, acetone, ethyl acetate, dimethyl carbonate, acetonitrile, dichloromethane, methyl ethyl ketone, methyl isobutyl ketone, 1-pentanol, methyl acetate, carbon tetrachloride, dimethyl sulfoxide, hexafluoroacetone, chlorobutanol, dimethyl sulfone, acetic acid, and cyclohexane.

Optionally, the mixture from step 2) may be filtered before lyophilization. The mixture in step 2) may be contained in vials. Optional aseptic vial filling may also be conducted before lyophilization. The vials can be sealed after lyophilization in step 3).

The drug solution obtained in step 1) may comprise around 0.05 mol/L, 0.10 mol/L, 0.15 mol/L, 0.20 mol/L, 0.30 mol/L, 0.40 mol/L, 0.50 mol/L of SDC-TRAP-0063 or a tautomer or a pharmaceutically acceptable salt thereof. The solution may have a pH of at least about 7, 8, 9, 10, 11, or 12.

The dry powders obtained in step 3) may comprise at least 20%, 25%, 50%, 75%, 90%, or 95% of the drug compound.

The dry powders can be resolved in a solvent to obtain a pharmaceutical composition to be administered to a patient. Such a pharmaceutical composition may have less than about 5.0%, 4.5%, 4.0%, 3.5%, 3.0%, 2.5%, 2.0%, 1.0%, 0.5%, or 0.1% of TBA by weight and at least about 25 mg/mL, 50 mg/mL, 75 mg/mL, 100 mg/mL, 125 mg/mL, 150 mg/mL, 200 mg/mL, 225 mg/mL, or 250 mg/mL of SDC-TRAP-0063, its tautomer, or its pharmaceutically acceptable salt. In some embodiments, the pharmaceutical composition may comprise between about 5% and about 0.1%, between about 4.0% and about 0.1%, between about 3.0% and about 0.1%, between about 2.0% and about 0.1%, between about 1.0% and about 0.1%, or between 0.5% and about 0.1% by weight of TBA.

In some embodiments, in step 2), the polyol or polyether is selected from the group consisting of PG, glycerol, and PEG. The concentration of the polyol or polyether in the mixture by weight may be between about 1% to about 10%, such as around 5% or 6%.

In some embodiments, in step 2), PEG is added to the drug solution. In one embodiment, 3-4% of PEG is added. PEG may be PEG400 (i.e., PEG-400), PEG-1000 (i.e., PEG-1000), PEG2000 (i.e., PEG-2000), or PEG-4000 (i.e., PEG-4000).

In some embodiments, the drug compound may be any pharmaceutical conjugate compound (SDC-TRAP compound) disclosed in WO 2017/151425, WO 2013/158644, WO 2015/038649, WO 2015/066053, WO 2015/116774, WO 2015/134464, WO 2015/143004, and WO 2015/184246, wherein the drug compound entraps TBA, forms a solvate with TBA, has a high pH, and/or limited water solubility.

### Pharmaceutical Compositions Comprising a Drug Compound

In one aspect, the present disclosure provides a pharmaceutical composition comprising a drug product that has a low amount of TBA, wherein TBA is utilized during the manufacturing of the drug product. The drug product in a lyophilized dry powder form may be dissolved in a solvent to obtain a pharmaceutical composition to be administered to a patient. The solvent may be any solvent suitable for administration to patients, such as water or saline. In some embodiments, such a pharmaceutical composition comprises less than about 5.0%, 4.5%, 4.0%, 3.5%, 3.0%, 2.5%, 2.0%, 1.0%, 0.5%, or 0.1% of TBA by weight. The drug compound in the pharmaceutical composition has a concentration of above about 0.01 mol/L, 0.02 mol/L, 0.03 mol/L, 0.04 mol/L, 0.05 mol/L, 0.1 mole/L, 0.15 mol/L or 0.2 mol/L. In some embodiments, the pharmaceutical composition may comprise between about 5% and about 0.1%, between about 4.0% and about 0.1%, between about 3.0% and about 0.1%, between about 2.0% and about 0.1%, between about 1.0% and about 0.1%, or between 0.5% and about 0.1% by weight of TBA.

In some embodiments, the drug compound is SDC-TRAP-0063, its tautomer, or its pharmaceutically acceptable salt. The pharmaceutical composition comprises less than about 5.0%, 4.5%, 4.0%, 3.5%, 3.0%, 2.5%, 2.0%, 1.0%, 0.5%, or 0.1% of TBA and at least about 25 mg/mL, 50 mg/mL, 75 mg/mL, 100 mg/mL, 125 mg/mL, 150 mg/mL, 200 mg/mL, 225 mg/mL, or 250 mg/mL of SDC-TRAP-0063, its tautomer, or its pharmaceutically acceptable salt. In some embodiments, the pharmaceutical composition may comprise between about 5% and about 0.1%, between about 4.0% and about 0.1%, between about 3.0% and about 0.1%, between about 2.0% and about 0.1%, between about 1.0% and about 0.1%, or between 0.5% and about 0.1% by weight of TBA.

### Excipients

The pharmaceutical composition may comprise other pharmaceutically acceptable excipients. A pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, Md., 2006; incorporated herein by reference in its entirety) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this invention.

In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical compositions.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, etc., and/or combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and VEEGUM® [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [TWEEN®20], polyoxyethylene sorbitan [TWEEN®60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g. polyoxyethylene monostearate [MYRJ®45], polyoxyethylene hydrogenated castor oil, polyethoxylated castor oil, polyoxymethylene stearate, and Kolliphor® (SOLUTOL®)), sucrose fatty acid esters, polyethylene glycol fatty acid esters (e.g. CREMOPHOR®), polyoxyethylene ethers, (e.g. polyoxyethylene lauryl ether [BRIJ®30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLUORINC®F 68, POLOXAMER®188, cetrimonium bromide, cetylpyridinium chloride, benzalkonium chloride, docusate sodium, etc. and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan); alginates; polyethylene oxide; polyethylene glycol; inorganic calcium salts; silicic acid; polymethacrylates; waxes; water; alcohol; etc.; and combinations thereof.

Exemplary preservatives may include, but are not limited to, antioxidants, chelating agents, antimicrobial preservatives, antifungal preservatives, alcohol preservatives, acidic preservatives, and/or other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Exemplary alcohol preservatives include, but are not limited to, ethanol, polyethylene glycol, phenol, phenolic compounds, bisphenol, chlorobutanol, hydroxybenzoate, and/or phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and/or phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL®115, GERMABEN®II, NEOLONE™, KATHON™, and/or EUXYL®.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and/or combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and/or combinations thereof.

Excipients such as cocoa butter and suppository waxes, coloring agents, coating agents, sweetening, flavoring, and/or perfuming agents can be present in the composition, according to the judgment of the formulator.

Exemplary bulking agents include mannitol, sucrose or one of the other disaccharides and these can be used in the composition.

### Methods of Using the Drug Compound Compositions

The drug compound composition with reduced TBA levels may be used in the treatment of a variety of different disease conditions. The specific disease conditions treatable by with the drug compounds are as varied as the types of drug compounds. Thus, disease conditions include cellular proliferative diseases, such as neoplastic diseases, autoimmune diseases, central nervous system or neurodegenerative diseases, cardiovascular diseases, hormonal abnormality diseases, infectious diseases, and the like.

By treatment is meant at least an amelioration of the symptoms associated with the disease condition afflicting the host, where amelioration is used in a broad sense to refer to at least a reduction in the magnitude of a parameter, e.g., symptom, associated with the pathological condition being treated, such as inflammation and pain associated therewith. As such, treatment also includes situations where the pathological condition, or at least symptoms associated therewith, are completely inhibited, e.g., prevented from happening, or stopped, e.g., terminated, such that the host no longer suffers from the pathological condition, or at least the symptoms that characterize the pathological condition.

Methods of use of the drug compounds extend beyond strict treatment of a disease. For example, the drug compounds may be used in a clinical or research setting to diagnose a subject, select a subject for therapy, select a subject for participation in a clinical trial, monitor the progression of a disease, monitor the effect of therapy, to determine if a subject should discontinue or continue therapy, to determine if a subject has reached a clinical end point, and to determine recurrence of a disease.

A variety of hosts are treatable according to the present disclosure. Generally such hosts are “mammals” or “mammalian,” where these terms are used broadly to describe organisms which are within the class Mammalia, including the orders carnivore (e.g., dogs and cats), rodentia (e.g., mice, guinea pigs, and rats), and primates (e.g., humans, chimpanzees, and monkeys). In many embodiments, the hosts will be humans.

The invention provides kits for treating a subject in need thereof comprising at least one drug compound and instruction for administering a therapeutically effective amount of the drug compound to the subject, thereby treating the subject. The invention also provides kits for imaging, diagnosing, and/or selecting a subject comprising at least one drug compound and instruction for administering an effective amount of the drug compound to the subject, thereby imaging, diagnosing, and/or selecting the subject.

### Administration

The compositions described herein contain an effective amount of a drug compound in a pharmaceutical carrier appropriate for administration to an individual in need thereof. The compositions may be administered by any route which results in a therapeutically effective outcome. These include, but are not limited to enteral, gastroenteral, epidural, oral, transdermal, epidural (peridural), intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection, (into the base of the penis), intravaginal administration, intrauterine, extra-amniotic administration, transdermal (diffusion through the intact skin for systemic distribution), transmucosal (diffusion through a mucous membrane), insufflation (snorting), sublingual, sublabial, enema, eye drops (onto the conjunctiva), or in ear drops. In specific embodiments, compositions may be administered in a way which allows them cross the blood-brain barrier, vascular barrier, or other epithelial barrier.

In some embodiments, the compositions are formulated for parenteral delivery, such as injection or infusion, in the form of a solution, suspension or emulsion. The compositions can be administered systemically, regionally or directly to the organ or tissue to be treated.

Parenteral formulations can be prepared as aqueous compositions using techniques is known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In some cases, an isotonic agent is included, for example, one or more sugars, sodium chloride, or other suitable agent known in the art.

Solutions and dispersions of the drug compound can be prepared in water or another solvent or dispersing medium suitably mixed with one or more pharmaceutically acceptable excipients including, but not limited to, surfactants, dispersants, emulsifiers, pH modifying agents, and combinations thereof.

Suitable surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine and lauryl sulfobetaine.

The compositions can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the drug compound.

The compositions are typically buffered to a pH of 3-8 for parenteral administration upon reconstitution. Suitable buffers include, but are not limited to, phosphate buffers, acetate buffers, and citrate buffers. If using 10% sucrose or 5% dextrose, a buffer may not be required.

Water soluble polymers are often used in compositions for parenteral administration. Suitable water-soluble polymers include, but are not limited to, polyvinylpyrrolidone, dextran, carboxymethylcellulose, and polyethylene glycol.

Sterile injectable solutions can be prepared by incorporating the drug compound in the required amount in the appropriate solvent or dispersion medium with one or more of the excipients listed above, as required, followed by filtered sterilization. Generally, dispersions are prepared by incorporating the various sterilized drug compounds into a sterile vehicle which contains the basic dispersion medium and the required other ingredients from those listed above.

Pharmaceutical compositions for parenteral administration can be in the form of a sterile aqueous solution or suspension of the drug compound. Acceptable solvents include, for example, water, Ringer's solution, phosphate buffered saline (PBS), and isotonic sodium chloride solution. The compositions may also be a sterile solution, suspension, or emulsion in a nontoxic, parenterally acceptable diluent or solvent such as 1,3-butanediol.

In some instances, the compositions are distributed or packaged in a liquid form. Alternatively, compositions for parenteral administration can be packed as a solid, obtained, for example by lyophilization of a suitable liquid formulation. The solid can be reconstituted with an appropriate carrier or diluent prior to administration.

Solutions, suspensions, or emulsions for parenteral administration may be buffered with an effective amount of buffer necessary to maintain a pH suitable for ocular administration. Suitable buffers are well known by those skilled in the art and some examples of useful buffers are acetate, borate, carbonate, citrate, and phosphate buffers.

Solutions, suspensions, or emulsions for parenteral administration may also contain one or more tonicity agents to adjust the isotonic range of the formulation. Suitable tonicity agents are well known in the art and some examples include glycerin, sucrose, dextrose, mannitol, sorbitol, sodium chloride, and other electrolytes.

Solutions, suspensions, or emulsions for parenteral administration may also contain one or more preservatives to prevent bacterial contamination of the ophthalmic preparations. Suitable preservatives are known in the art, and include polyhexamethylenebiguanidine (PHMB), benzalkonium chloride (BAK), stabilized oxychloro complexes (otherwise known as Purite®), phenylmercuric acetate, chlorobutanol, sorbic acid, chlorhexidine, benzyl alcohol, parabens, thimerosal, and mixtures thereof.

Solutions, suspensions, or emulsions for parenteral administration may also contain one or more excipients known art, such as dispersing agents, wetting agents, and suspending agents.

### Dosing

The present invention provides methods comprising administering the pharmaceutical compositions to a subject in need thereof. The compositions may be administered to a subject using any amount and any route of administration effective for preventing or treating or imaging a disease, disorder, and/or condition (e.g., a disease, disorder, and/or condition relating to working memory deficits). The exact amount required will vary from subject to subject, depending on the species, age, and general condition of the subject, the severity of the disease, the particular composition, its mode of administration, its mode of activity, and the like.

The compositions are typically formulated in dosage unit form for ease of administration and uniformity of dosage. It will be understood, however, that the total daily usage of the compositions may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In some embodiments, compositions in accordance with the present invention may be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, from about 25 mg/kg to about 50 mg/kg, from about 50 mg/kg to about 100 mg/kg, from about 100 mg/kg to about 125 mg/kg, from about 125 mg/kg to about 150 mg/kg, from about 150 mg/to about 175 mg/kg, from about 175 mg/kg to about 200 mg/kg, from about 200 mg/kg to about 250 mg/kg of subject body weight per day, one or more times a day, to obtain the desired therapeutic, diagnostic, prophylactic, or imaging effect. The desired dosage may be delivered three times a day, two times a day, once a day, every other day, every third day, every week, every two weeks, every three weeks, or every four weeks. In some embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used.

As used herein, a “split dose” is the division of single unit dose or total daily dose into two or more doses, e.g, two or more administrations of the single unit dose. As used herein, a “single unit dose” is a dose of any therapeutic administered in one dose/at one time/single route/single point of contact, i.e., single administration event. As used herein, a “total daily dose” is an amount given or prescribed in 24 hr period. It may be administered as a single unit dose. In one embodiment, the compositions are administered to a subject in split doses.

### Definitions

The articles “a,” “an,” and “the” are used herein to refer to one or to more than one (i.e. to at least one) of the grammatical object of the article unless otherwise clearly indicated by contrast. By way of example, “an element” means one element or more than one element.

The term “including” is used herein to mean, and is used interchangeably with, the phrase “including but not limited to.”

The term “or” is used herein to mean, and is used interchangeably with, the term “and/or,” unless context clearly indicates otherwise.

The term “such as” is used herein to mean, and is used interchangeably, with the phrase “such as but not limited to.”

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The recitation of a listing of chemical group(s) in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

As used herein, the term “subject” refers to human and non-human animals, including veterinary subjects. The term “non-human animal” includes all vertebrates, e.g., mammals and non-mammals, such as non-human primates, mice, rabbits, sheep, dog, cat, horse, cow, chickens, amphibians, and reptiles. In a preferred embodiment, the subject is a human and may be referred to as a patient.

As used herein, the terms “treat,” “treating” or “treatment” refer, preferably, to an action to obtain a beneficial or desired clinical result including, but not limited to, alleviation or amelioration of one or more signs or symptoms of a disease or condition, diminishing the extent of disease, stability (i.e., not worsening) state of disease, amelioration or palliation of the disease state, diminishing rate of or time to progression, and remission (whether partial or total), whether detectable or undetectable. “Treatment” can also mean prolonging survival as compared to expected survival in the absence of treatment. Treatment does not need to be curative.

A “therapeutically effective amount” is that amount sufficient to treat a disease in a subject. A therapeutically effective amount can be administered in one or more administrations.

By “diagnosing” and the like, as used herein, refers to a clinical or other assessment of the condition of a subject based on observation, testing, or circumstances for identifying a subject having a disease, disorder, or condition based on the presence of at least one indicator, such as a sign or symptom of the disease, disorder, or condition. Typically, diagnosing using the method of the invention includes the observation of the subject for multiple indicators of the disease, disorder, or condition in conjunction with the methods provided herein. Diagnostic methods provide an indicator that a disease is or is not present. A single diagnostic test typically does not provide a definitive conclusion regarding the disease state of the subject being tested.

The terms “administer,” “administering” or “administration” include any method of delivery of a pharmaceutical composition or agent into a subject's system or to a particular region in or on a subject. In certain embodiments of the invention, an agent is administered intravenously, intramuscularly, subcutaneously, intradermally, intranasally, orally, transcutaneously, or mucosally. In a preferred embodiment, an agent is administered intravenously. Administering an agent can be performed by a number of people working in concert. Administering an agent includes, for example, prescribing an agent to be administered to a subject and/or providing instructions, directly or through another, to take a specific agent, either by self-delivery, e.g., as by oral delivery, subcutaneous delivery, intravenous delivery through a central line, etc.; or for delivery by a trained professional, e.g., intravenous delivery, intramuscular delivery, intratumoral delivery, etc.

As used herein, the term “survival” refers to the continuation of life of a subject which has been treated for a disease or condition, e.g., cancer. The time of survival can be defined from an arbitrary point such as time of entry into a clinical trial, time from completion or failure or an earlier treatment regimen, time from diagnosis, etc.

As used herein, the term “recur” refers to the re-growth of tumor or cancerous cells in a subject in whom primary treatment for the tumor has been administered. The tumor may recur in the original site or in another part of the body. In one embodiment, a tumor that recurs is of the same type as the original tumor for which the subject was treated. For example, if a subject had an ovarian cancer tumor, was treated and subsequently developed another ovarian cancer tumor, the tumor has recurred. In addition, a cancer can recur in or metastasize to a different organ or tissue than the one where it originally occurred.

As used herein, the terms “identify” or “select” refer to a choice in preference to another. In other words, to identify a subject or select a subject is to perform the active step of picking out that particular subject from a group and confirming the identity of the subject by name or other distinguishing feature.

As used herein, the term “benefit” refers to something that is advantageous or good, or an advantage. Similarly, the term “benefiting,” as used herein, refers to something that improves or advantages. For example, a subject will benefit from treatment if they exhibit a decrease in at least one sign or symptom of a disease or condition (e.g., tumor shrinkage, decrease in tumor burden, inhibition or decrease of metastasis, improving quality of life (“QOL”), if there is a delay of time to progression (“TTP”), if there is an increase of overall survival (“OS”), etc.), or if there is a slowing or stopping of disease progression (e.g., halting tumor growth or metastasis, or slowing the rate of tumor growth or metastasis). A benefit can also include an improvement in quality of life, or an increase in survival time or progression free survival.

The terms “cancer” or “tumor” are well known in the art and refer to the presence, e.g., in a subject, of cells possessing characteristics typical of cancer-causing cells, such as uncontrolled proliferation, immortality, metastatic potential, rapid growth and proliferation rate, decreased cell death/apoptosis, and certain characteristic morphological features. Cancer cells are often in the form of a solid tumor. However, cancer also includes non-solid tumors, e.g., blood tumors, e.g., leukemia, wherein the cancer cells are derived from bone marrow. As used herein, the term “cancer” includes pre-malignant as well as malignant cancers. Cancers include, but are not limited to, acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, Burkitt's lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, heavy chain disease, hemangioblastoma, hepatoma, hepatocellular cancer, hormone insensitive prostate cancer, leiomyosarcoma, liposarcoma, lung cancer, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin, and uterus, lymphoid malignancies of T-cell or B-cell origin, leukemia, lymphoma, medullary carcinoma, medulloblastoma, melanoma, meningioma, mesothelioma, multiple myeloma, myelogenous leukemia, myeloma, myxosarcoma, neuroblastoma, non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer, and Wilms' tumor. Other cancers include primary cancer, metastatic cancer, oropharyngeal cancer, hypopharyngeal cancer, liver cancer, gall bladder cancer, bile duct cancer, small intestine cancer, urinary tract cancer, kidney cancer, urothelium cancer, female genital tract cancer, uterine cancer, gestational trophoblastic disease, male genital tract cancer, seminal vesicle cancer, testicular cancer, germ cell tumors, endocrine gland tumors, thyroid cancer, adrenal cancer, pituitary gland cancer, hemangioma, sarcoma arising from bone and soft tissues, Kaposi's sarcoma, nerve cancer, ocular cancer, meningial cancer, glioblastomas, neuromas, neuroblastomas, Schwannomas, solid tumors arising from hematopoietic malignancies such as leukemias, metastatic melanoma, recurrent or persistent ovarian epithelial cancer, fallopian tube cancer, primary peritoneal cancer, gastrointestinal stromal tumors, colorectal cancer, gastric cancer, melanoma, glioblastoma multiforme, non-squamous non-small-cell lung cancer, malignant glioma, epithelial ovarian cancer, primary peritoneal serous cancer, metastatic liver cancer, neuroendocrine carcinoma, refractory malignancy, triple negative breast cancer, HER2-amplified breast cancer, nasopharageal cancer, oral cancer, biliary tract, hepatocellular carcinoma, squamous cell carcinomas of the head and neck (SCCHN), non-medullary thyroid carcinoma, recurrent glioblastoma multiforme, neurofibromatosis type 1, CNS cancer, liposarcoma, leiomyosarcoma, salivary gland cancer, mucosal melanoma, acral/lentiginous melanoma, paraganglioma, pheochromocytoma, advanced metastatic cancer, solid tumor, triple negative breast cancer, colorectal cancer, sarcoma, melanoma, renal carcinoma, endometrial cancer, thyroid cancer, rhabdomysarcoma, multiple myeloma, ovarian cancer, glioblastoma, gastrointestinal stromal tumor, mantle cell lymphoma, and refractory malignancy.

“Solid tumor,” as used herein, is understood as any pathogenic tumor that can be palpated or detected using imaging methods as an abnormal growth having three dimensions. A solid tumor is differentiated from a blood tumor such as leukemia. However, cells of a blood tumor are derived from bone marrow; therefore, the tissue producing the cancer cells is a solid tissue that can be hypoxic.

“Tumor tissue” is understood as cells, extracellular matrix, and other naturally occurring components associated with the solid tumor.

As used herein, the term “isolated” refers to a preparation that is substantially free (e.g., 50%, 60%, 70%, 80%, 90% or more, by weight) from other proteins, nucleic acids, or compounds associated with the tissue from which the preparation is obtained.

The term “sample” as used herein refers to a collection of similar fluids, cells, or tissues isolated from a subject. The term “sample” includes any body fluid (e.g., urine, serum, blood fluids, lymph, gynecological fluids, cystic fluid, ascetic fluid, ocular fluids, and fluids collected by bronchial lavage and/or peritoneal rinsing), ascites, tissue samples (e.g., tumor samples) or a cell from a subject. Other subject samples include tear drops, serum, cerebrospinal fluid, feces, sputum, and cell extracts. In one embodiment, the sample is removed from the subject. In a particular embodiment, the sample is urine or serum. In another embodiment, the sample does not include ascites or is not an ascites sample. In another embodiment, the sample does not include peritoneal fluid or is not peritoneal fluid. In one embodiment, the sample comprises cells. In another embodiment, the sample does not comprise cells. Samples are typically removed from the subject prior to analysis. However, tumor samples can be analyzed in the subject, for example, using imaging or other detection methods.

The term “control sample,” as used herein, refers to any clinically relevant comparative sample, including, for example, a sample from a healthy subject not afflicted with cancer, a sample from a subject having a less severe or slower progressing cancer than the subject to be assessed, a sample from a subject having some other type of cancer or disease, a sample from a subject prior to treatment, a sample of non-diseased tissue (e.g., non-tumor tissue), a sample from the same origin and close to the tumor site, and the like. A control sample can be a purified sample, protein, and/or nucleic acid provided with a kit. Such control samples can be diluted, for example, in a dilution series to allow for quantitative measurement of analytes in test samples. A control sample may include a sample derived from one or more subjects. A control sample may also be a sample made at an earlier time point from the subject to be assessed. For example, the control sample could be a sample taken from the subject to be assessed before the onset of the cancer, at an earlier stage of disease, or before the administration of treatment or of a portion of treatment. The control sample may also be a sample from an animal model, or from a tissue or cell lines derived from the animal model, of the cancer. The level in a control sample that consists of a group of measurements may be determined, e.g., based on any appropriate statistical measure, such as, for example, measures of central tendency including average, median, or modal values.

As used herein, the term “obtaining” is understood herein as manufacturing, purchasing, or otherwise coming into possession of.

“Elevated” or “lower” refers to a patient's value of a marker relative to the upper limit of normal (“ULN”) or the lower limit of normal (“LLN”) which are based on historical normal control samples. As the level of the marker present in the subject will be a result of the disease, and not a result of treatment, typically a control sample obtained from the patient prior to onset of the disease will not likely be available. Because different labs may have different absolute results, values are presented relative to that lab's upper limit of normal value (ULN).

“Determining” as used herein is understood as performing an assay or using a diagnostic method to ascertain the state of someone or something, e.g., the presence, absence, level, or degree of a certain condition, biomarker, disease state, or physiological condition.

“Prescribing” as used herein is understood as indicating a specific agent or agents for administration to a subject.

The terms “administer,” “administering” or “administration” can include any method of delivery of a pharmaceutical composition or agent into a subject's system or to a particular region in or on a subject. In certain embodiments of the invention, an Hsp90 inhibitor is administered intravenously, intramuscularly, subcutaneously, intradermally, intranasally, orally, transcutaneously, or mucosally. In a preferred embodiment, an agent is administered intravenously. Administering can be performed by a number of people working in concert. Administering an agent includes, for example, prescribing an agent to be administered to a subject and/or providing instructions, directly or through another, to take a specific agent, either by self-delivery, e.g., as by oral delivery, subcutaneous delivery, intravenous delivery through a central line, etc.; or for delivery by a trained professional, e.g., intravenous delivery, intramuscular delivery, intratumoral delivery, etc.

“Pharmaceutical conjugate” refers to a non-naturally occurring molecule that includes a binding moiety (e.g., an Hsp90-targeting moiety) associated with an effector moiety, where these two components may also be covalently bonded to each other either directly or through a linking group.

The term “drug” or “drug compound” refers to any active agent that affects any biological process. Active agents that are considered drugs for purposes of this application are agents that exhibit a pharmacological activity. Examples of drugs include active agents that are used in the prevention, diagnosis, alleviation, treatment or cure of a disease condition.

By “pharmacologic activity” is meant an activity that modulates or alters a biological process so as to result in a phenotypic change, e.g., cell death, cell proliferation etc.

By “pharmacokinetic property” is meant a parameter that describes the disposition of an active agent in an organism or host.

By “half-life” is meant the time for one-half of an administered drug to be eliminated through biological processes, e.g., metabolism, excretion, etc.

The term “efficacy” refers to the effectiveness of a particular active agent for its intended purpose, i.e., the ability of a given active agent to cause its desired pharmacologic effect.

### EXAMPLES

The following examples, which are briefly summarized and then discussed in turn below, are offered by way of illustration and not by way of limitation.

### Example 1: Synthesis of SDC-TRAP-0063

### SDC-TRAP-0063

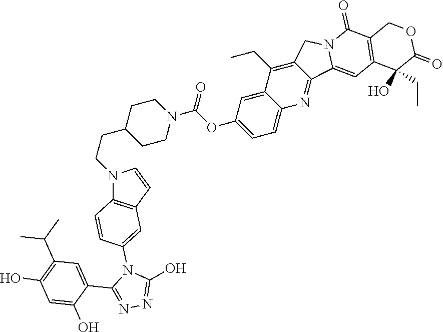 [SMILES: CCc1c2c(nc3ccc(OC(=O)N4CCC(CCn5ccc6cc(-n7c(O)nnc7-c7cc(C(C)C)c(O)cc7O)ccc65)CC4)cc13)-c1cc3c(c(=O)n1C2)COC(=O)[C@]3(O)CC]

((S)-4,11-diethyl-4-hydroxy-3,14-dioxo-3,4,12,14-tetrahydro-1H-pyrano[3′,4′:6,7]indolizino[1,2-b]quinolin-9-yl 4-(2-(5-(3-(2,4-dihydroxy-5-isopropylphenyl)-5-hydroxy-4H-1,2,4-triazol-4-yl)-1H-indol-1-yl)ethyl)piperidine-1-carboxylate) or its tautomer.

A synthesis scheme of the synthesis of SDC-TRAP-0063 is provided in Example 6 of PCT Application No. PCT/US2013/036783.

### Example 2. Lyophilization Composition Investigation

SDC-TRAP-0063 synthesized in Example 1 was dissolved in an alkaline co-solvent system comprising 30% (by volume) tert-butanol (TBA) and water to obtain a drug solution. Various polyols, such as PEG, glycerol and propylene glycol (PG), were added to the drug solution in vials prior to the standard lyophilization cycle (freezing→annealing→primary drying→secondary drying). Dry powder drug products comprising SDC-TRAP-0063 sodium were obtained. TBA weight percentages in the dry powders were measured by gas chromatography (GC-HS) (Table 1).

TABLE 1
TBA reduction after adding diols or triols as excipients

| Formulation of Drug Solution Pre-Lyophilization | | | | | Testing of Lyophilized | |
|---|---|---|---|---|---|---|
| SDC-TRAP-0063 | | | | | Drug Product | |
| Concentration | | % PEG | % | | | Residual |
| (mg/mL) | % TBA | 400 | Glycerol | % PG | pH | TBA |
| 100 | 30 | 0 | 0 | 0 | 10.1-10.3 | 7% avg |
| 100 | 30 | 4 | | | 10.1 | 2.0% |
| 100 | 30 | 3 | | | 10.1 | 3.6% |
| 100 | 30 | | 4 | | 10.2 | 1.2% |
| 100 | 30 | | 2 | | 10.2 | 5.2% |
| 100 | 30 | | 1 | | 10.2 | 7.1% |
| 100 | 30 | | | 4 | 10.1 | 1.5% |
| 100 | 30 | | | 2 | 10.2 | 4.1% |

As shown in Table1, adding PEG400, glycerol, and PG reduced residual TBA levels after the standard lyophilization cycle. The desirable concentration is 3-4% of PEG, propylene glycol or glycerol.

It was also found that stability declined with addition of some of the excipients: Propylene glycol addition resulted in faster decay of SDC-TRAP-0063 sodium. PEG-400 had no impact on the stability of SDC-TRAP-0063 sodium.

### Example 3. PEG-400 and Glycerol Concentration Optimization

Various concentrations of PEG-400 and glycerol and various lyophilization conditions were studied for optimized TBA removed. Results are shown in Table 2.

TABLE 2
TBA reduction after adding PEG-400 or glycerol as excipients

| Formulation of Drug Solution Pre-Lyophilization | | | | | Testing of Lyophilized | | |
|---|---|---|---|---|---|---|---|
| SDC-TRAP-0063 | | | | | Drug Product | | |
| Concentration | | % PEG | % | | | Residual | Residual |
| (mg/mL) | % TBA | 400 | Glycerol | Conditions | pH | TBA | Moisture |
| 100 | 30 | 0 | 0 | Standard | 10.1-10.3 | 7% avg | |
| | | | | cycle | | | |
| 100 | 30 | 4 | | Standard | 10.1 | 2.7% | 0.26% |
| 100 | 30 | 3.5 | | cycle | 10.0 | 3.3% | |
| 100 | 30 | 3.5 | | No secondary | 10.1 | 3.2% | |
| | | | | drying | | | |
| 100 | 30 | | 4 | Standard | 10.1 | 1.8% | |
| 100 | 30 | | 3.5 | cycle | 10.1 | 3.3% | |
| 100 | 30 | | 3 | | 10.1 | 3.8% | |
| 100 | 30 | | 3 | No secondary | 10.1 | 4.0% | |
| | | | | drying | | | |

It was found that glycerol is a more efficient excipient for TBA reduction, but it results in a slightly worse stability profile. 4% of PEG-400 is sufficient to reduce TBA to 2.5-2.7%, and the stability profile is acceptable. Therefore, 4% PEG-400 was selected for further optimization.

Further, the efficiency of TBA removal is driven by composition, and is not impacted significantly by lyophilization cycle parameters. Secondary drying has a minor effect on the final TBA levels.

### Example 4. PEG Composition Optimizations

### NaOH Load and PEG-400 Concentration

The effects of adding NaOH and/or benzyl alcohol (BnOH) and varying PEG-400 concentrations were investigated in this study. From the results shown in Table 3, 4% PEG is a primary choice. NaOH and/or BnOH load has a minor impact on TBA removal.

TABLE 3
TBA reduction after adding NaOH and/or BnOH with PEG-400

| Formulation of Drug Solution Pre-Lyophilization | | | | | Testing of Lyophilized | |
|---|---|---|---|---|---|---|
| SDC-TRAP-0063 | | | | | Drug Product | |
| Concentration | | % PEG | BnOH | NaOH | | Residual |
| (mg/mL) | % TBA | 400 | (mg/vial) | (g/100 g) | pH | TBA |
| 100 | 30 | 0 | 0 | 0 | 10.1-10.3 | 7% avg |
| 100 | 30 | 4 | | 5.5 | 9.9 | 2.2% |
| 100 | 30 | 4 | 0.1 | 5.5 | 10.0 | 2.4% |
| 100 | 30 | 4 | | 5 | 9.5 | 1.9% |
| 100 | 30 | 3 | | 5.5 | 9.9 | 4.1% |
| 100 | 15 | 3 | | 5.5 | 10.0 | 3.3% |

### Molecular Weight of PEG and Other Alcohols

PEG with larger molecular weight and mannitol were used as excipients and their impact on TBA levels were determined. As the results in Table 4 show, molecular weight of PEG has almost no impact on TBA removal. Lower amount of TBA in the pre-lyophilization drug solution leads to less TBA in the lyophilized drug product. However, 15% TBA and 4 or 6% Mannitol still did not work as well as adding PEG for TBA removal.

TABLE 4
TBA reduction after adding PEG or Mannitol

| Formulation of Drug Solution Pre-Lyophilization | | | | | | Testing of Lyophilized | |
|---|---|---|---|---|---|---|---|
| SDC-TRAP-0063 | | | | | | Drug Product | |
| Concentration | | % PEG | % PEG | % PEG | % | | Residual |
| (mg/mL) | % TBA | 1000 | 2000 | 4000 | Mannitol | pH | TBA |
| 100 | 30 | 0 | 0 | 0 | 0 | 10.1-10.3 | 7% avg |
| 100 | 30 | 4 | | | | 9.7 | 2.3% |
| 100 | 30 | | 4 | | | 9.9 | 2.2% |
| 100 | 30 | | | 4 | | 9.8 | 2.3% |
| 100 | 15 | | | | 6 | 9.8 | 6.6% |
| 100 | 15 | | | | 4 | 9.8 | 6.0% |

Additional increase of PEG-400 concentration up to 6% allowed further reduction of TBA to almost non-detectable level. As the results in Table 5 show, efficiency of TBA removal. Mannitol was added to the composition as a bulking agent to prevent cake collapse.

TABLE 5
TBA reduction after adding PEG and Mannitol as a bulking agent

| Formulation of Drug Solution Pre-Lyophilization | | | | Testing of |
|---|---|---|---|---|
| SDC-TRAP-0063 | | | | Lyophilized |
| Concentration | | | | Drug Product |
| (mg/mL) | % TBA | % PEG 400 | % Mannitol | Residual TBA |
| 100 | 30 | 4 | 5 | 0.54 |
| 100 | 30 | 4 | 7.5 | 1.36 |
| 100 | 30 | 4 | 10 | 2.22 |
| 100 | 30 | 5 | 5 | 0.11 |
| 100 | 30 | 5 | 7.5 | 0.26 |
| 100 | 30 | 5 | 10 | 0.74 |
| 100 | 30 | 6 | 5 | 0.05* |
| 100 | 30 | 6 | 7.5 | 0.09* |
| 100 | 30 | 6 | 10 | 0.16 |

*Below the limit of quantitation of the method

1. A process of producing a lyophilized drug product comprising the steps of:
1). dissolving a drug compound in a co-solvent system comprising tert-butanol (TBA) and at least one other solvent to obtain a drug solution;
2). adding at least one polyol to the drug solution to obtain a mixture; and
3). conducting lyophilization to obtain the lyophilized drug product.

2. The process of claim 1, wherein the other solvent is selected from the group consisting of water, ethanol, n-propanol, n-butanol, isopropanol, methanol, acetone, ethyl acetate, dimethyl carbonate, acetonitrile, dichloromethane, methyl ethyl ketone, methyl isobutyl ketone, 1-pentanol, methyl acetate, carbon tetrachloride, dimethyl sulfoxide, hexafluoroacetone, chlorobutanol, dimethyl sulfone, acetic acid, and cyclohexane.

3. The process of claim 1, wherein the polyol in step 2) is a diol or a triol.

4. The process of claim 1, wherein the polyol in step 2) is selected from propylene glycol, glycerol and polyethylene glycol (PEG).

5. The process of claim 4, wherein the polyol in step 2) is PEG.

6. The process of claim 5, wherein the polyol in step 2) is PEG400, PEG1000, PEG2000 or PEG4000.

7. The process of claim 5, wherein about 3%-6% PEG is added.

8. The process of claim 1, wherein the lyophilized drug product in step 3) comprises at least 20%, 25%, 50%, 75%, 90%, or 95% by weight of the drug compound.

9. The process of claim 1, wherein the lyophilized drug product in step 3) comprises less than about 5.0%, 4.5%, 4.0%, 3.5%, 3.0%, 2.5%, or 2.0%, 1.0%, 0.5%, or 0.1% by weight of TBA.

10. The process of claim 1, wherein the drug solution in step 1) comprises about 0.05 mol/L, 0.10 mol/L, 0.15 mol/L, 0.20 mol/L, 0.30 mol/L, 0.40 mol/L, 0.50 mol/L of the drug compound.

11. The process of claim 1, wherein the drug solution in step 1) has a pH of at least about 7, 8, 9, 10, 11, or 12.

12. The process of claim 1, wherein the drug compound forms a solvate with TBA.

13. The process of claim 1, wherein the drug compound is ((S)-4,11-diethyl-4-hydroxy-3,14-dioxo-3,4,12,14-tetrahydro-1H-pyrano[3′,4′:6,7]indolizino[1,2-b]quinolin-9-yl 4-(2-(5-(3-(2,4-dihydroxy-5-isopropylphenyl)-5-hydroxy-4H-1,2,4-triazol-4-yl)-1H-indol-1-yl)ethyl)piperidine-1-carboxylate), its tautomer, or its pharmaceutically acceptable salt.

14. A pharmaceutical composition comprising the drug product produced from the process in claim 1 and at least one pharmaceutically acceptable excipient.

15. The pharmaceutical composition of claim 14, wherein the drug compound is ((S)-4,11-diethyl-4-hydroxy-3,14-dioxo-3,4,12,14-tetrahydro-1H-pyrano[3′,4′:6,7]indolizino[1,2-b]quinolin-9-yl 4-(2-(5-(3-(2,4-dihydroxy-5-isopropylphenyl)-5-hydroxy-4H-1,2,4-triazol-4-yl)-1H-indol-1-yl)ethyl)piperidine-1-carboxylate) or its tautomer.

16. The pharmaceutical composition of claim 14, wherein the pharmaceutical composition is in liquid form and comprises a solvent.

17. The pharmaceutical composition of claim 16, wherein the pharmaceutical composition comprises less than about 5.0%, 4.5%, 4.0%, 3.5%, 3.0%, 2.5%, 2.0%, 1.0%, 0.5%, or 0.0.1% by weight of TBA.

18. The pharmaceutical composition of claim 16, comprising at least about at least about 25 mg/mL, 50 mg/mL, 75 mg/mL, 100 mg/mL, 125 mg/mL, 150 mg/mL, 200 mg/mL, 225 mg/mL, or 250 mg/mL of the drug compound.

19. A method of treating a patient comprising administering the pharmaceutical composition of claim 14.

20. The method of claim 19, wherein the drug compound is ((S)-4,11-diethyl-4-hydroxy-3,14-dioxo-3,4,12,14-tetrahydro-1H-pyrano[3′,4′:6,7]indolizino[1,2-b]quinolin-9-yl 4-(2-(5-(3-(2,4-dihydroxy-5-isopropylphenyl)-5-hydroxy-4H-1,2,4-triazol-4-yl)-1H-indol-1-yl)ethyl)piperidine-1-carboxylate), its tautomer, or its pharmaceutically acceptable salt.

21. The method of claim 19, wherein the patient has cancer.
